(19) United States
(12) Patent Application Publication
(10) Pub. No.: US 2020/0113577 A1
(43) Pub. Date: Apr. 16, 2020

(54) VASCULAR TREATMENT APPARATUS WITH REMOTE OPERATING MODULE

(71) Applicant: Merit Medical Systems, Inc., South Jordan, UT (US)

(72) Inventors: Carl J. Wisnosky, Woodstock Valley, CT (US); Arnold Feinberg, Stoughton, MA (US); John P. Marano, JR., Madison, CT (US)

(21) Appl. No.: 16/600,193

(22) Filed: Oct. 11, 2019

Related U.S. Application Data
(60) Provisional application No. 62/745,023, filed on Oct. 12, 2018.
(60) Provisional application No. 62/745,035, filed on Oct. 12, 2018.

(51) Int. Cl. A61B 17/12

(52) U.S. Cl. CPC A61B 17/12186; A61B 2017/1205; A61B 17/12031; A61B 17/12109

(57) ABSTRACT

An apparatus for performing a vascular treatment is disclosed. In some embodiments, the apparatus comprises two modules that are maintained in separate zones of an operating environment. The apparatus includes an elongate, intraluminal member that is shaped and dimensioned for passage through blood vessels of a subject. The intraluminal member has a proximal end and a distal end. A vein wall disruptor for degrading the integrity of the inner wall of a vessel is housed within a sheath of the intraluminal member. The disruptor includes at least one, but may include several, prongs. The distal ends of the prongs contact or communicate with the inner wall of a blood vessel. The disruptor can include a contacting tip designed to damage the interior of the vessel as the disruptor is pulled proximally therealong. The apparatus further includes an automatic injector to inject a sclerosant into the blood vessel.

### RELATED APPLICATIONS

This application claims priority to U.S. Provisional Application No. 62/745,023, filed on Oct. 12, 2018 and titled “Auto Injection Device For Vascular Ablation Apparatus and Methods,” and to U.S. Provisional Application No. 62/745,035, filed on Oct. 12, 2018 and titled “Vascular Treatment Apparatus,” each of which is hereby incorporated by reference in its entirety.

### TECHNICAL FIELD

The present disclosure relates generally to the field of vascular treatment. More specifically, the present disclosure relates to devices and methods used to disrupt and occlude diseased veins and lymphatic vessels.

### BRIEF DESCRIPTION OF THE DRAWINGS

FIG. 1 is a perspective view of an embodiment of a vascular treatment device.

FIG. 2 is a perspective view of a portion of the vascular treatment device of FIG. 1.

FIG. 3 is a perspective view of an embodiment of a reservoir.

FIG. 4 is a perspective view of another embodiment of a reservoir.

FIG. 4A is a front view of the reservoir of FIG. 4.

FIG. 4B is a side view of the reservoir of FIG. 4.

FIG. 5 is perspective view of another embodiment of a reservoir.

FIG. 5A is a front view of the reservoir of FIG. 5 with lateral compression.

FIG. 5B is a front view of the reservoir of FIG. 5 with radial compression.

FIG. 6 is a perspective view of an automatic injector device.

FIG. 7A is a top view of another embodiment of a vascular treatment device.

FIG. 7B is a perspective view of the vascular treatment device of FIG. 7A.

FIG. 8 is a perspective exploded view of another embodiment of a vascular treatment device.

FIG. 9A is a side view of another embodiment of a vascular treatment device.

FIG. 9B is a partial cut-away perspective view of the vascular treatment device of FIG. 9A.

FIG. 10A is a side view of another embodiment of a vascular treatment device.

FIG. 10B is a partial cut-away perspective view of the vascular treatment device of FIG. 10A.

FIG. 11 is a side view of a portion of an embodiment of a disruptor.

FIG. 12 is a side view of a portion of another embodiment of a disruptor.

### DETAILED DESCRIPTION

Sclerotherapy may be utilized in the treatment of blood vessel malformations, and similar problems in body systems such as the lymphatic system. Sclerotherapy may be used for treating various vein conditions, such as varicose veins, reticular veins, spider veins of the leg, and also some fine facial veins.

Sclerotherapy can be used to treat these conditions by instigating vascular fibrosis and obliteration in response to irreversible endothelial cellular destruction and/or exposure of the underlying subendothelial cell layer. In some instances, this destruction can be caused by the injection of a chemical agent such as a sclerosant into the vein.

Methods and devices for treating the vascular system may also include the use of devices which physically displace the endothelium of the vein in order to augment the effect of the chemical agent. In some embodiments, these devices may be multifunctioning in that an operator may control both the introduction of the sclerosant and the actuation of physical means for disrupting the inner wall of a vessel.

A device is disclosed in the present disclosure for occluding a vein that generally utilizes a physical disruption component which is assisted by a chemical agent to degrade the vein and force its collapse. The device may be configured to be introduced through minimally invasive surgical techniques.

In certain embodiments, the device may include an elongate, hollow intraluminal member that is shaped and dimensioned for passage through blood vessels of a subject. A vein wall disruptor for degrading the integrity of the inner wall of a vessel can be housed within the distal end of intraluminal member. The disruptor can include at least one prong, but may include several prongs, the distal end of which may be configured to contact the inner wall of a blood vessel. For example, the disruptor can include a contacting tip configured to damage the interior of the vessel as the disruptor is rotated and/or pulled proximally therealong. A chemical agent may also aid the disruptor in degrading the inner wall of the vessel. In some of such embodiments, the intraluminal member may act as a conduit to a source of the chemical agent thereby allowing the agent to flow into the interior of the vessel.

In one embodiment the disruptor can be driven in a rotating motion to damage and/or degrade the inner wall of the vessel. The disruptor can be linked to a motor by a drive shaft in order to impart rotational motion thereto. The drive shaft may be housed within the intraluminal member. The motor may be reversible and connected to a control circuit in order to randomize the direction of rotation.

In some embodiments, certain components of the device may be disposed remotely from other components. For example, some components may be in a handle assembly while other components may be in an control module. The control module may be connected to the handle assembly by a conduit. The conduit may comprise one or more of a fluid supply tube, a drive shaft, and power/control wiring. For instance, the control module may have a housing that contains a reservoir for a chemical agent that is in communication with the elongated hollow intraluminal member via the fluid supply tube. The control module may further include an automatic injector device for infusing the chemical agent through the supply tube to the intraluminal member. An actuator for activating a motor to rotate the disruptor, via a drive shaft, may also be included in the control module, as may a power source and various control circuitry.

In certain embodiments, a proximal end of the elongate, hollow intraluminal member may be disposed within the handle assembly and protrude from a distal end thereof. The handle assembly may comprise controls which are coupled to the control module so as to actuate the disruptor and control the flow of the chemical agent. In certain embodiments, the handle assembly can include a manual actuator for deploying the disruptor (e.g., retracting the intraluminal member and/or extending the disruptor) such that the disruptor contacts the inner wall of a vessel. The manual actuator can also be used to return the disruptor to its undeployed, or housed state, within the intraluminal member.

The following description and examples illustrate certain embodiments in detail. Those of skill in the art will recognize that there are variations and modifications of this invention that are encompassed by its scope. Accordingly, the description of one embodiment should not be deemed to limit the scope of the device as described here.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the intraluminal member of the vessel occluding device, the proximal end of the intraluminal member refers to the end nearest the handle and the distal end refers to the opposite end, the end inserted into the vessel. Thus, if at one or more points in a procedure a physician changes the orientation of the intraluminal member, as used herein, the term “proximal end” always refers to the handle end of the intraluminal member (even if the distal end is temporarily closer to the practitioner).

“Fluid” is used in its broadest sense, to refer to any fluid, including both liquids and gases as well as solutions, compounds, suspensions, etc., which generally behave as fluids.

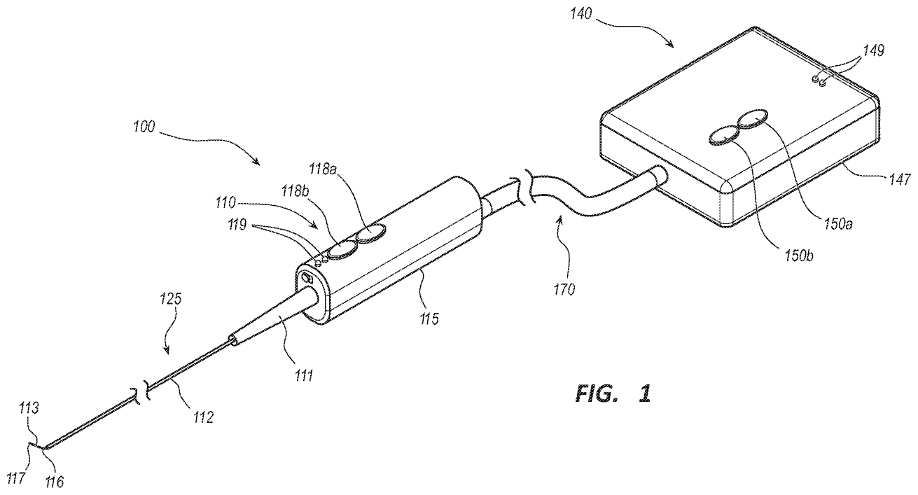

FIGS. 1-11 illustrate different views of several vascular treatment devices and related components. In certain views each device may be coupled to, or shown with, additional components not included in every view. Further, in some views only selected components are illustrated to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1-6 depict one embodiment of a vascular treatment device 100. In the illustrated embodiment, the vascular treatment device 100 generally includes three groups of components; and each group may have numerous subcomponents and parts. The three component groups are: a handle assembly 110, an control module 140, and a conduit 170.

FIG. 1 depicts a perspective view of one embodiment of exemplary components of the vascular treatment device 100. These components can be configured to provide a range of functionalities to the vascular treatment device 100. As shown in FIG. 1, the vascular treatment device 100 generally includes a handle assembly 110 and a control module 140 that are coupled together by a conduit 170. The control module 140 is depicted as being remote from the handle assembly 110, with the conduit 170 extending between the handle assembly 110 and the control module 140. As explained in greater detail below, each of the handle assembly 110 and control module 140 can include features configured for stimulating vascular ablation and/or for delivering a chemical agent, for example a liquid or gas, to the interior of a vessel of a patient.

As depicted in FIG. 1, the handle assembly 110 may include a body 115, a strain relief 111, and an intraluminal member 125. The body 115 may be configured to be ergonomically efficient, allowing an operator to manipulate the handle assembly 110 with simple motions during a medical procedure. An outer surface of the body 115 may be constructed from a pliable material allowing the operator to gain a better grip on the body 115. The body 115 may be formed of any suitable rigid or semi-rigid material. For example, the body 115 may be formed from polymeric materials such as polycarbonate, polypropylene, polyethylene, acrylic, styrene, etc., or from metals, such as stainless steel, titanium, etc., or combinations thereof. The body 115 may be transparent, translucent, or opaque. The body 115 may comprise a top half and a bottom half that may be coupled together using any suitable technique such as snap fit, welding, gluing, bonding, etc.

The strain relief tube or guard 111 is shown to extending distally from a distal end of the body 115 and surrounding a proximal portion of the intraluminal member 125. The strain relief 111 may be configured to protect and/or stabilize the intraluminal member 125 where it exits the body 115. For example, the strain relief 111 may prevent the intraluminal member 125 from kinking that may prevent fluid from flowing through the intraluminal member 125. The strain relief 111 may be tapered such that a proximal end has a larger diameter than a distal end. The strain relief 111 may be formed from any suitable pliable material, such as polymeric materials like silicone, thermoplastic elastomer, rubber, neoprene, etc., or combinations thereof.

The depicted embodiment of FIG. 1 shows the intraluminal member 125 extending distally from the body 115 of the handle assembly 110. The intraluminal member 125 may be configured to be inserted into and moved along a vessel of a patient. The intraluminal member 125 may include a sheath or outer sheath 112 having a lumen extending therethrough. The sheath 112 can have a variety of inner and outer diameters. For example, the sheath 112 can have an inner diameter ranging from approximately 0.022 inches to 0.048 inches and an outer diameter ranging from approximately 0.025 inches to 0.051 inches. In some embodiments, the outer diameter of the sheath 112 can be in the range that is consistent with an inner diameter of standard needles or vascular sheaths. For example, the sheath 112 can be sized and shaped to be insertable through a standard needle or vascular sheath having an inner diameter ranging from approximately 0.0035 inches to 0.0160 inches, from approximately 0.0160 inches to 0.0420 inches, from approximately 0.0420 inches to 0.0630 inches, or from approximately 0.0115 inches to 0.0630 inches. The sheath 112 may also include external markings at regular intervals which may guide the user to monitor the insertion or removal speed and/or depth of the sheath 112 into or out of a vessel.

The sheath 112 may be formed from any suitable material. For example, the sheath 112 may be formed from polymeric materials such as polyurethane, fluorinated ethylene propylene, polyether block amide, polypropylene, polyethylene, etc., or combinations thereof. In some embodiments, the sheath 112 may comprise a radiopacifying agent, such as barium sulfate, bismuth trioxide, zirconium dioxide, etc. In other embodiments, the sheath 112 may comprise radiopaque markers or bands disposed at various locations along a length of the sheath 112. For example, the radiopaque markers or bands may be disposed at about 5 cm, 10 cm, 20 cm, etc. intervals along the length of the sheath 112.

A disruptor 113 may be displaceably disposed within the sheath 112. The disruptor 113 may include an elongate wire 116 and a distal disruptor tip 117 disposed at a distal end of the elongate wire 116. The disruptor 113 can comprise a variety of materials and geometric configurations. In some embodiments, the disruptor 113 can be configured to facilitate injection of liquid into a patient. For example, the elongate wire 116 can be channeled to allow fluid flow to a desired injection point as the fluid flows through intraluminal member 125. In certain embodiments, the elongate wire 116 may be hollow to allow for fluid flow through the elongate wire 116 and distal disruptor tip 117 to a desired injection point at the end of the intraluminal member. In another embodiment, a diameter of the elongate wire 116 can be sized such that an annular space between an outside surface of the elongate wire 116 and an inside surface of the sheath 112 allows fluid to flow through the intraluminal member 125 (e.g., within the annular space) and to the desired injection point at the distal end of the intraluminal member. The disruptor 113 may be formed from any suitable material, such as stainless steel, nickel titanium alloy, titanium, etc.

The distal disruptor tip 117 may include at least one prong configured to bias the distal disruptor tip 117 towards or against an inner wall of the vessel. In some embodiments, the distal disruptor tip 117 may include two, three, four, five, or more prongs. The disruptor 113, including the distal disruptor tip 117, is configured to to communicate, engage, irritate, and/or damage the inner wall of the vessel. In further embodiments, the disruptor 113, including the distal disruptor tip 117, is further configured such that it does not break through the entirety of the inner wall the vessel.

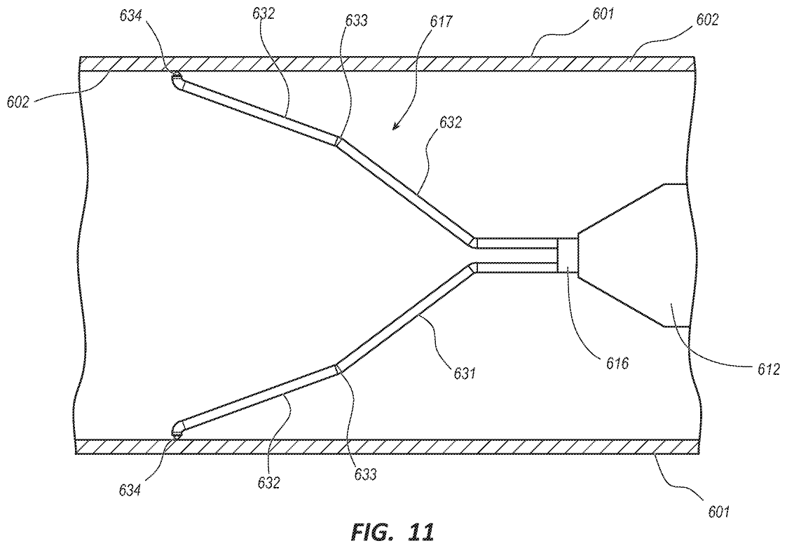

An example of a distal disruptor tip 617 that may be employed with the vascular treatment device 100 is shown in FIG. 11, wherein the distal disruptor tip 617 is positioned in a vessel 601. The distal disruptor tip 617 is attached to the distal end of a disruptor wire 616 and is housed within a sheath 612 prior to deployment. As shown in FIG. 11 the distal disruptor tip 617 comprises a plurality of flexible prongs 631 that each include a plurality of substantially linear segments 632 disposed between a plurality of bent segments 633. A head 634 of the distal disruptor tip 617 is located at a distal end of each prong 631 and may be configured to mechanically degrade the inner wall 602 of the vessel 601. The flexible prongs 631 may be configured to bias radially outward when the distal disruptor tip 617 is deployed from the sheath 612 such that the head 634 of each prong 631 is biased towards or against the inner wall 602 of the vessel 601.

The head 634 located at the distal end of each prong 631 may have a wide variety of configurations, depending on the intended use. A shape of the head 634 may be “atraumatic,” meaning that it may be shaped such that during insertion of the distal disruptor tip 617, the head 634 causes little or no spasm or damage to the vessel. For example, the head 634 may have a hemispheric shape. The hemispheric shaped head 634 may be textured or mechanically or chemically altered to create a roughened surface. Other atraumatic tips may include a head 634 having a full radius, or a J-curved shape, or simply a curved shape. In other embodiments, the head 634 may be “aggressive” and be bent or curved so that it scrapes the vessel wall 602. The head 634 may have a flat end with a sharp edge around the flat end. An aggressive head 634 may also be created by beveling an edge to create a sharp point. The aggressive head 634 may have a cutting blade, like a shark's fin. The head 634 may be roughened to make the head 634 cut more aggressively and/or cause the blood vessel wall to spasm.

In general, the degradation of the inner wall 602 of the vessel 601 by the distal disruptor tip 617 may be caused by a radial outwardly directed force to communicate, engage, irritate, and/or damage but not break through the vessel wall 602. This can be accomplished by imparting rotational motion to the distal disruptor tip 617 such that the distal disruptor tip 617 rotates and contacts the vessel wall. This can also be accomplished by imparting rotational motion to the distal disruptor tip 617 to impart a fluid shear force rather than, or in addition to, a radial force. The fluid shear force may be generated as the distal disruptor tip 617 is rotated adjacent the vessel wall 602, but not contacting the vessel wall 602. As the distal disruptor tip 617 is rotated, blood or other fluid may be dragged by the head 634 resulting in a rotational shear force on the vessel wall 602. Alternatively or in combination with rotation of the distal disruptor tip 617, the distal disruptor tip 617 may be drawn linearly (e.g., proximally) against the vessel wall 602 to irritate and damage but not break through the vessel wall 602. The linear movement of the distal disruptor tip 617 can be accomplished manually by displacing the distal distal disruptor tip 617 in a proximal direction via movement of the handle, or with use of a motor in the handle or control module 140.

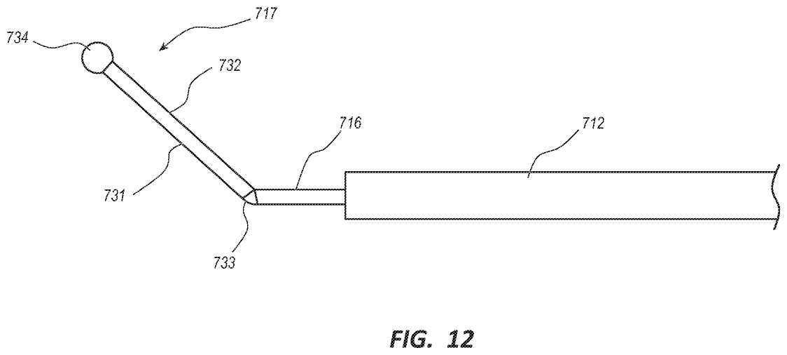

Another example of a distal disruptor tip 717 that may be employed with the vascular treatment device 100 is shown in FIG. 12. The distal disruptor tip 717 is attached to a distal end of a disruptor wire 716 and is housed within a sheath 712 prior to deployment. As shown in FIG. 12 the distal disruptor tip 617 comprises a flexible prong 731 that includes a linear segment 732 disposed distal of a bent segment 733. A head 734 (e.g., sphere) of the distal disruptor tip 717 is located at a distal end of the prong 731 and may be configured to mechanically degrade an inner wall of a vessel. The flexible prong 731 may be configured to bias radially outward when the distal disruptor tip 717 is deployed from the sheath 712 such that the head 734 is biased towards or against the inner wall of the vessel.

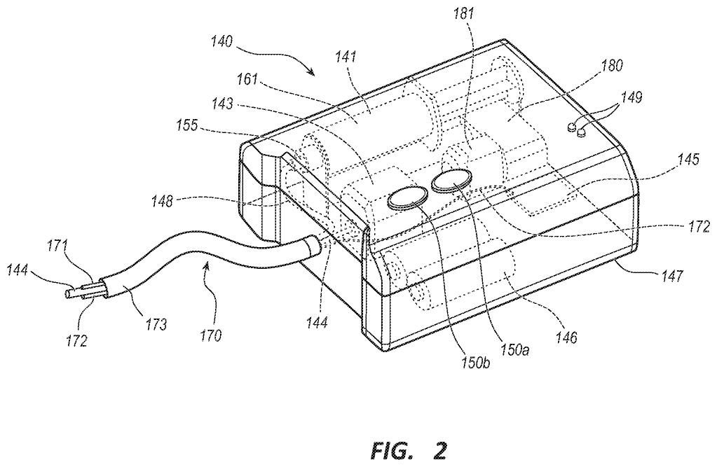

With continued reference to FIGS. 1 and 2, the vascular treatment device 100 can include the remote or control module 140. The control module 140 can include one or more of a housing 147, a fluid reservoir 141, a motor 143, a power source 146, an automatic injector 180, and a control circuit 145 for regulating the functions of the various components of the control module 140. The control circuit 145 may be configured as a printed circuit board. The control circuit 145 may be coupled to the motor 143 and can be configured to control, for example, speed and direction of rotation of the motor 143. Similarly, the control circuit 145 can control the automatic injector 180 to control the rate of injection of a fluid from the reservoir 141. In some embodiments, the control circuit 145 can be linked to the handle assembly 110 and, in particular, control buttons 118a, 118b and status indicators 119 by control wiring 172 such that certain components of the control module 140 can be controlled by the operator from the handle assembly 110. In another embodiment, the control circuit 145 can be linked to control buttons 150a, 150b and status indicators 149 of the control module 140 by the control wiring 172 such that certain components of the control module 140 can be controlled remotely from the handle assembly 110 by the control module 140. In still another embodiment, the control circuit 145 can be linked to the control buttons 118a, 118b, 150a, 150b and the status indicators 119, 149 by the control wiring 172 such that certain components of the control module 140 can be controlled from both the handle assembly 110 and the control module 140. Alternatively, the control buttons 118a, 118b can be linked wirelessly or via Bluetooth. The power source 146 may comprise a rechargeable battery, a non-rechargeable battery, or an outlet plug to directly supply power from an outlet.

The reservoir 141 may be in fluid communication with the intraluminal member 125 through a fluid channel 148. The fluid channel 148 may include a fluid transfer tubing 171 which is coupled to an outlet 155 of the reservoir 141 at one end and the intraluminal member 125 at another end. The fluid channel 148 may further include the lumen of the sheath 112 such that the fluid is configured to flow from the reservoir 141, through the fluid transfer tubing 171, through the lumen of the sheath 112, and out of the distal end of the sheath 112. As the reservoir 141 is filled and the fluid transferred through the fluid channel 148, internal surfaces of the reservoir 141 and the fluid channel 148 and the fluid are maintained in a sterile state. In one embodiment the reservoir 141 holds a chemical agent, such as a sclerosant, that is injected into the vessel while the distal disruptor tip 117 is degrading the inner wall of the vessel. The reservoir 141 may comprise a chamber 161 having the outlet 155.

In some embodiments, the reservoir 141 can be acted on by the automatic injector 180 to force fluid from the outlet 155. The automatic injector 180 can be operated electro-mechanically, with gas pressure, mechanically, hydraulically, or using a fluid switch device. Actuation of the reservoir 141 to displace the fluid can be through a variety of ways (e.g., translating a plunger 153 within the reservoir 141; using a side action pressure plate to compress the reservoir 141; using gas pressure to compress the reservoir 141; using a sliding constrictive device traveling parallel to the centerline, etc.). The automatic injector 180 may also include a fluid sensor, not shown, that can detect a fluid level in the reservoir 141 and stop the automatic injector 180 when a required fluid volume has been delivered. The fluid level detection can be sensed electrically, mechanically, fluid flow or on a time basis. In the depicted embodiment, the automatic injector 180 can comprise a solenoid valve 181 that actuates the reservoir 141 and is controlled by the control circuit 145 that in turn communicates with control buttons 118a, 118b, 150a, 150b.

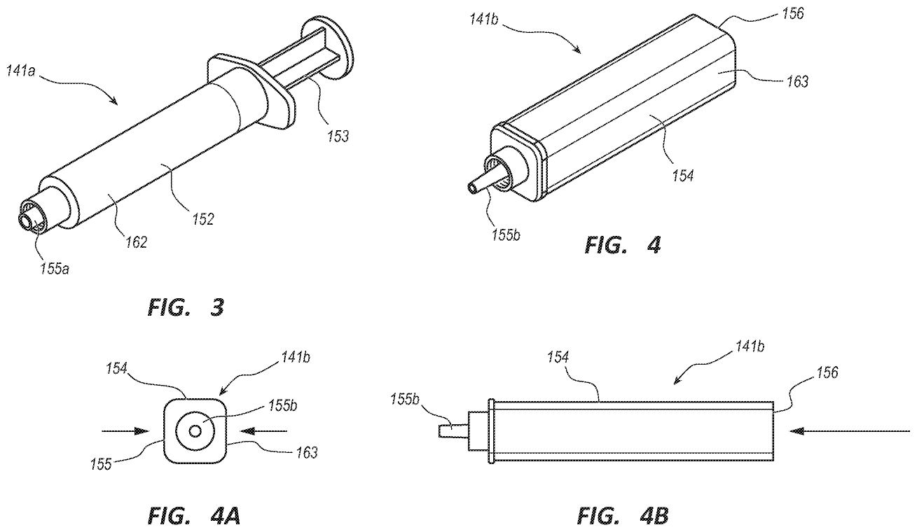

Several exemplary embodiments of the reservoir 141 are shown in FIGS. 3-6. As shown in FIG. 3, a reservoir 141a can comprise a syringe 152. The syringe 152 may comprise a barrel 162 and a plunger 153. The syringe 152 can be placed within the automatic injector 180 such that the automatic injector 180 acts on the plunger 153 to force the fluid out of outlet 155a.

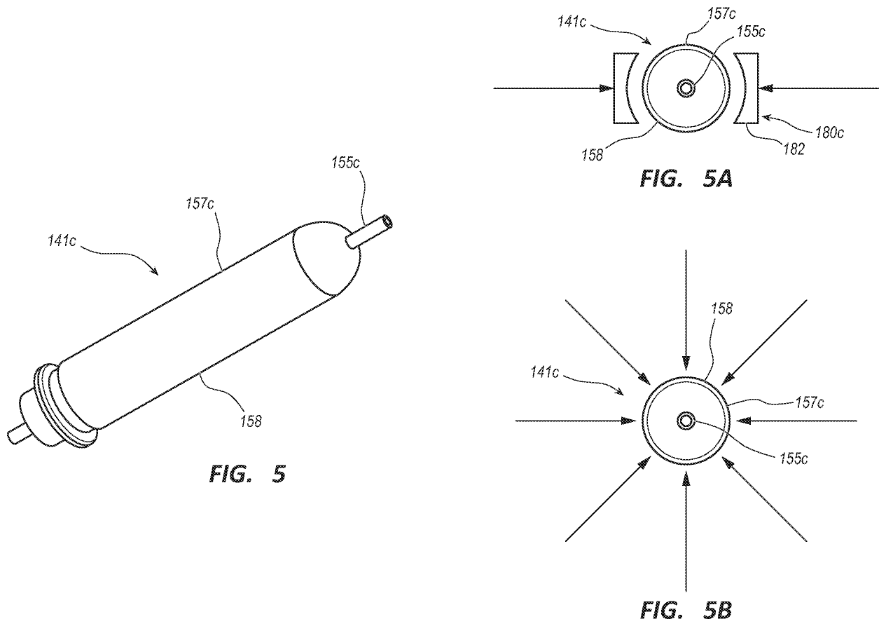

As shown in FIGS. 4-4B, a reservoir 141b comprises a cartridge 154. As shown in FIG. 4A, the cartridge 154 can have movable side walls 163 that can be compressed by a lateral force as indicated by the arrows. When the reservoir 141b is laterally compressed, fluid may be forced out of the outlet 155b. Alternatively and as shown in FIG. 4B, the cartridge 154 can include a movable rear wall 156 that is displaced by a distally directed force, as indicated by the arrow, to force the fluid from outlet 155b.

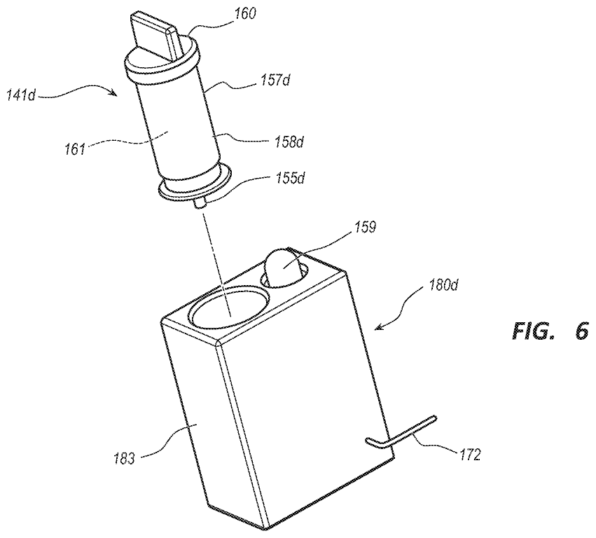

FIGS. 5-5B show yet another embodiment of a reservoir 141c comprising a soft sided cartridge 157c. The cartridge 157c includes a pliable wall 158 made from a pliable material that can readily by compressed. As shown in FIG. 5A, an automatic injector 180c can include lateral actuators 182 that can be actuated from the sides of cartridge 157, as indicated by the arrows, to compress it and force fluid from the outlet 155c. Alternatively and as shown in FIG. 5B, the cartridge 157c can be circumferentially constricted, as indicated by the arrows, to force the fluid from outlet 155c.

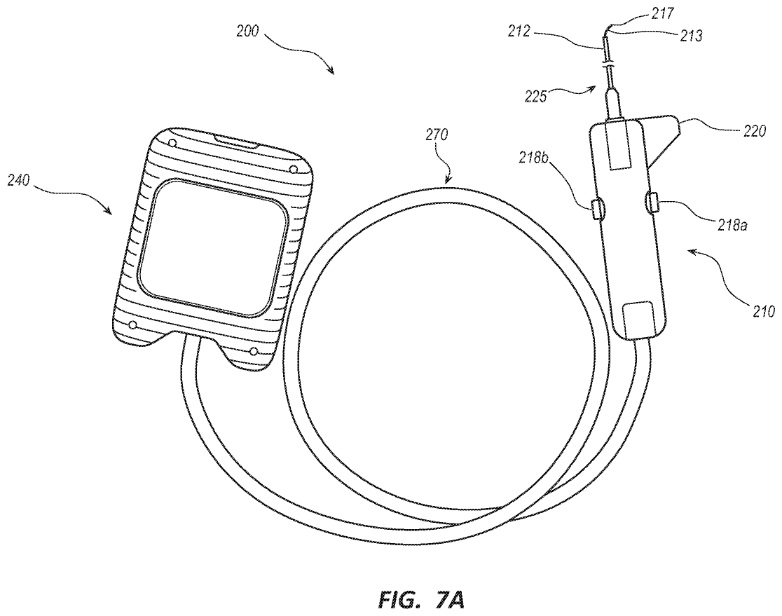

FIG. 6 shows an integrated automatic injector 180d and reservoir 141d that may be incorporated into the control module 140 of the vascular treatment device 100. The integrated automatic injector 180d and reservoir 141d of FIG. 6 comprise a housing 183. An insertable cartridge 157d is mountable within the housing 183 and includes an outlet 155d and a locking head 160. Once inserted into the housing 183, the locking head 160 may be twisted to lock the cartridge 157d into place. This also aligns a gas inlet with a gas line, not shown in the drawings. The gas line is in communication with a pressurized gas source 159, such as a CO₂ or compressed air cartridge. The integrated automatic injector 180d and the fluid reservoir 141d communicate with the control circuit 145 via the control wiring 172 such that a valve on the gas source 159 is actuated and compressed gas is released into the cartridge 157d. In one embodiment, the compressed gas forces an inner rear wall of the cartridge 157d downward, forcing the fluid in the cartridge 157d out of the outlet 155d. The inner rear wall is sealed so as not to permit the compressed gas from the gas source 159 into a chamber 161 containing the fluid. Other types of reservoirs can also be used.

As depicted in FIGS. 1 and 2, the control module 140 may be operably coupled to the handle assembly 110 by the elongate conduit 170. The conduit 170 may be configured to allow remote control of the motor 143 and the automatic injector 180. This can allow the control module 140 to be positioned away from a sterile procedure field and interface with the control module 140 to be performed by a person not manipulating the handle assembly 110. This can also allow for a lighter weight handle assembly 110, as many components are disposed in the control module 140. In some embodiments, the conduit 170 may allow the handle assembly 110 to be disposed of following a single use while the control module 140 may be used for multiple procedures (e.g., by uncoupling the handle assembly 110 from the control module 140 after use).

In the depicted embodiment, the conduit 170 may comprise an outer sheath or covering 173 and house one or more of the fluid transfer tubing 171, the control wiring 172, and a drive shaft 144. The fluid transfer tubing 171 may be in fluid communication with the reservoir 141 and the intraluminal member 125. The drive shaft 144 can be connected at its proximal end to the motor 143 and at its distal end to the disruptor 113 for imparting a rotational motion to the distal disruptor tip 117. The drive shaft 144 may include e.g., a core comprising a wire rope or coil and a covering comprising a steel liner, a steel and cloth braided material, and an elastomeric cover. Other types of drive shafts 144 can also be used. The control wiring 172 may be connected to the control circuit 145 at its proximal end and to the control buttons 118a, 118b and status indicators 119 of the handle assembly 110 at its distal end.

In certain embodiments, activation of the motor 143 can cause rotation of the disruptor 113. The control circuit 145 can be programmed to generate a desired speed of rotation of the motor 143 and consequently the drive shaft 144 and the disruptor 113. In one embodiment, an operator may vary the speed of the motor 143 (e.g., by pressing the control button 118a or 150a repeatedly to vary the speed). The motor 143 can be configured to rotate the drive shaft 144 and the disruptor 113 up to approximately 100 rpm, up to 500 rpm, up to 1,000 rpm, or up to 5,000 rpm. Some embodiments of the vascular treatment device 100 further comprise at least one feedback feature, such as a built-in RPM display.

In some embodiments, the components of the control module 140 may be alternatively disposed in the handle assembly 110. For example, the motor 143 may be disposed in the handle assembly 110 and directly coupled to the disruptor 113. For another example, the automatic injector 180 may be alternatively disposed in the handle assembly 110. Any combination of positioning of the components of the control module 140 in the handle assembly 110 is contemplated for this disclosure.

In operating the vascular treatment device 100, the operator may create an incision or use an access sheath and insert the elongate intraluminal member 112 into the vessel that is targeted for treatment. The operator may position the distal end of the intraluminal member 125 at the place where the treatment of the vessel will commence. The disruptor 112 can then be deployed. For example, the operator can push the control button 118a or 150a to activate a motor 143 to linearly displace either the disruptor 113 or sheath 112 to expose the distal disruptor tip 117 from the sheath 112. This exposes the distal disruptor tip 117 and forces the distal disruptor tip 117 towards, against, or to communicate with the inner wall of the targeted vessel. The operator can then initiate a degrading of the inner wall of the targeted vessel by engaging or scraping it. In some embodiments, the inner wall of the targeted vessel is scraped when the handle assembly 110 is displaced proximally resulting in the distal disruptor tip 117 longitudinally scraping the inner wall of the targeted vessel. In other embodiments, the inner wall of the targeted vessel is scraped when the handle assembly 110 is displaced proximally and manually rotated. In still other embodiments, the operator may push control button 118a or 150a to activate rotation of the motor 143 and the distal disruptor tip 117. The operator can also proximally displace the handle assembly 110 manually to longitudinally scrape the inner wall simultaneously if desired. Prior to, simultaneously with, or subsequent to scraping of the inner wall of the targeted vessel, the operator may press the control button 118b or 150b to activate the automatic injector 180 and start the flow of the sclerosant through the fluid channel 148 and out of the intraluminal member 125.

Once the procedure is completed, the operator can optionally return the distal disruptor tip 117 into its undeployed or housed position (e.g., within the intraluminal member). The operator can then remove the intraluminal member 125 from the vessel. The handle assembly 110 having been in the sterile zone can be disposed of. The control module 140, however, can be preserved outside the sterile zone and thus can be refurbished and re-used.

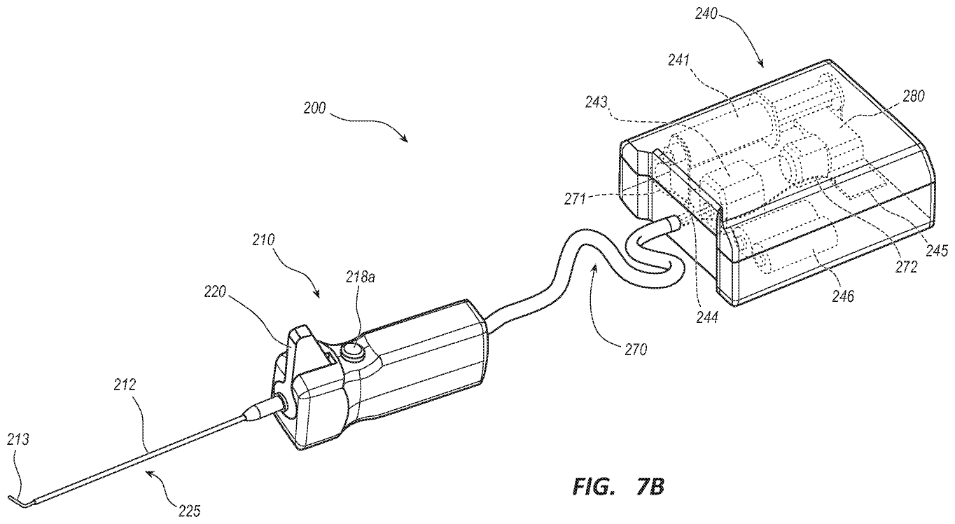

FIGS. 7A and 7B depict an embodiment of a vascular treatment device 200 that resembles the vascular treatment device 100 described above in certain respects. Accordingly, like features are designated with like reference numerals, with the leading digit incremented to “2.” For example, the embodiment depicted in FIGS. 7A and 7B includes a control module 240 that may, in some respects, resemble the control module 140 of FIGS. 1 and 2. Relevant disclosure set forth above regarding similarly identified features may not be repeated hereafter. Moreover, specific features of the vascular treatment device 100 and related components shown in FIGS. 1-6 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the vascular treatment device 200 and related components depicted in FIGS. 7A and 7B Any suitable combination of the features, and variations of the same, described with respect to the vascular treatment device 100 and related components illustrated in FIGS. 1-6 can be employed with the vascular treatment device 200 and related components of FIGS. 7A and 7B, and vice versa. This pattern of disclosure applies equally to further embodiments depicted in subsequent figures and described hereafter, wherein the leading digits may be further incremented.

FIGS. 7A and 7B illustrate an embodiment of a vascular treatment device 200. The vascular treatment device 200 may include a handle assembly 210, a control module 240, and an elongate conduit 270. As depicted, the handle assembly 210 includes a cartridge 220. The cartridge 220 may include an intraluminal member 225 extending from a distal end of the cartridge 220. The intraluminal member 225 may include a disruptor 213 disposed within a sheath 212. The cartridge 220 may be slidingly coupleable to the handle assembly 210. When the cartridge 220 is coupled to the handle assembly 210, the sheath 212 may be displaced proximally such that a distal disruptor tip 217 of the disruptor 213 extends beyond a distal end of the sheath 212. In another embodiment, when the cartridge 220 is coupled to the handle assembly 210, the disruptor 213 can be displaced distally such that the distal disruptor tip 217 of the disruptor 213 extends beyond the distal end of the sheath 212.

The handle assembly 210 may also include control buttons 218a and 218b. The control button 218a may activate a motor 243 when depressed. In other embodiments, the control button 218a may be configured to control a speed and a rotational direction of the motor 243. The control button 218b may activate the automatic injector 280 to cause fluid, such as a sclerosant, to remain sterile flow from the reservoir 241, through the intraluminal member 225, and out of the distal end of the intraluminal member 225.

As illustrated in FIG. 7B, the control module 240 may include a motor 243, an automatic injector 280, a reservoir 241, a control circuit 245, and a power source 246. The power source 246 may include one or more rechargeable or non-rechargeable batteries. In certain embodiments, the power source 246 may connect to a wall outlet. The power source 246 may be electrically connected to the control circuit 245. The control circuit 245 may be in the form of a printed circuit board. The control circuit 245 may be electrically connected to the motor 243, the automatic injector 280, and the control buttons 218a, 218b via a control wiring 272. The motor 243 may be configured to rotate in both directions. The automatic injector 280 may be configured to receive any suitable reservoir 241 and displace the fluid contained within the reservoir 241.

The conduit 270 may operably connect the control module 240 to the handle assembly 210, allowing the control module 240 to be positioned remotely from the handle assembly 210. The conduit 270 may comprise a drive shaft 244, a fluid transfer tubing 271, and the control wiring 272, all of which extend longitudinally within the conduit 270. A proximal end of the drive shaft 244 may be connected to the motor 243 and a distal end connected to a proximal end of the disruptor 213. A proximal end of the fluid transfer tubing 271 may be in fluid communication with the reservoir 241 and a distal end may be in fluid communication with a proximal end of the intraluminal member 225. A proximal end of the control wiring 272 may be connected to the control circuit 245 and a distal end connected to the control buttons 218a, 218b.

In operating the vascular treatment device 200, the operator may create an incision or use an access sheath and insert the elongate intraluminal member 225 of the cartridge 220 into a vessel that is targeted for treatment. The operator may position the distal end of the intraluminal member 212 at the place where the treatment of the vessel will commence and deploy the disruptor 213. For example, the operator can slidingly couple the cartridge 220 into the handle assembly 210 causing proximal displacement of the intraluminal member 212 relative to the disruptor 213. This exposes a distal disruptor tip 217 and forces the distal disruptor tip 217 towards, against, or to communicate with an inner wall of the targeted vessel. In another embodiment, the disruptor 213 may be displaced distally relative to the intraluminal member 212 when the cartridge 220 is coupled to the handle assembly 210.

The operator may initiate degrading of the inner wall of the targeted vessel by engaging or scraping it. In some embodiments, the inner wall of the targeted vessel may be scraped when the handle assembly 210 is drawn proximally, resulting in the distal disruptor tip 217 longitudinally scraping the inner wall. In other embodiments, the inner wall of the targeted vessel can be scraped when the handle assembly 210 is drawn proximally and manually rotated. In still other embodiments, the operator may push the control button 118a to activate rotation of the motor 243 and the distal disruptor tip 217. The operator can also simultaneously proximally displace the handle assembly 210 to longitudinally scrape the inner wall. Prior to, simultaneously with, or subsequent to scraping of the inner wall of the targeted vessel, the operator may press the control button 218b to activate the automatic injector 280 and start the flow of the sclerosant through the fluid transfer tubing 271 and out of the distal end of the intraluminal member 225.

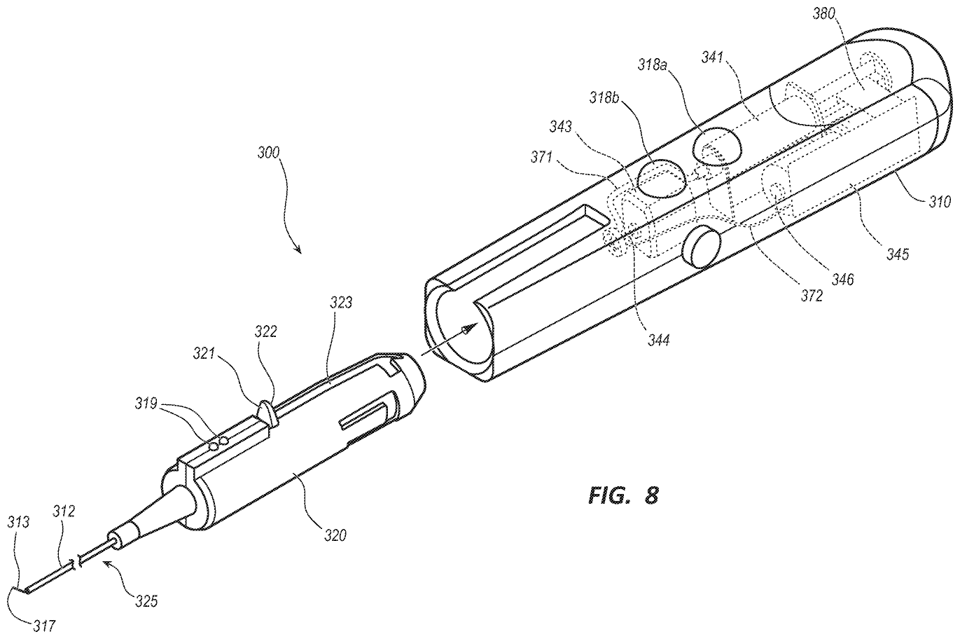

FIG. 8 illustrates another embodiment of a vascular treatment device 300. The vascular treatment device 300 is generally an integration of the handle assembly 210 and the control module 240 of the vascular treatment device 200 as previously described. The vascular treatment device 300 may include a handle assembly 310 and a cartridge 320. The cartridge 320 may include an intraluminal member 325 extending from a distal end of the cartridge 320. The intraluminal member 325 may include a disruptor 313 disposed within a sheath 312. The cartridge 320 may be slidingly coupleable to the handle assembly 310. The cartridge 320 may include a slider 321. The slider 321 may include an exterior grip portion 322 extending outwardly from a slot 323 disposed in the cartridge 320. The slider 321 may be coupled to a proximal end of the sheath 312 such that when the slider 321 is displaced from a distal position to a proximal position within the slot 323, the sheath 312 is displaced proximally relative to the disruptor 313 to expose a distal disruptor tip 317 of the disruptor 313. Following the procedure, the operator can optionally move the slider 321 from the proximal position to the distal position such that the distal disruptor tip 317 is no longer exposed.

In other embodiments, the slider 321 may be coupled to a proximal end of the disruptor 313 such that when the slider 321 is displaced from a proximal position to a distal position within the slot 323, the disruptor 313 is displaced distally relative to the sheath 312 to expose the distal disruptor tip 317 of the disruptor 313. In such embodiments, at the end of a procedure the operator can optionally move the slider 321 from the distal position to the proximal position such that the distal disruptor tip 317 is no longer exposed. The cartridge 320 may also include status indicators 319.

As depicted in the illustrated embodiment, the handle assembly 310 may include a motor 343, an automatic injector 380, a reservoir 341, a control circuit 345, and a power source 346. The power source 346 may include one or more rechargeable or non-rechargeable batteries. The power source 346 may be electrically connected to the control circuit 345. The control circuit 345 may be in the form of a printed circuit board. The control circuit 345 may be electrically connected to the motor 343, the automatic injector 380, control buttons 318a, 318b and the status indicators 319 of the cartridge 320 via a control wiring 372. The motor 343 may be operably coupled to a proximal end of the disruptor 313 via a drive shaft 344 and configured to rotate the disruptor 313 in both directions. The automatic injector 380 may be configured to receive any suitable reservoir 341 and displace the fluid contained within the reservoir 341. The reservoir 341 may be loaded into the handle assembly 310 during manufacture or loaded by an operator at a time of use. The reservoir 341 may be in fluid communication with the intraluminal member 325 via a fluid transfer tubing 371.

In operating the vascular treatment device 300, the operator may create an incision or use an access sheath and insert the elongate intraluminal member 325 of the cartridge 320 into a vessel that is targeted for treatment. The operator may position a distal end of the intraluminal member 325 at the place where the treatment of the vessel will commence, and deploy the disruptor 313. For example, the, operator can slidingly couple the cartridge 320 into the handle assembly 310. In some embodiments, the cartridge 320 may be coupled to the handle assembly 310 prior to insertion of the intraluminal member 325 into the vessel. The slider 321 can be displaced proximally to displace the sheath 312 proximally relative to the disruptor 313. This exposes the distal disruptor tip 317 and forces the distal disruptor tip 317 towards, against, or to communicate with an inner wall of the targeted vessel. In another embodiment, the slider 321 may be displaced distally to displace the disruptor 313 distally relative to the sheath 312.

The operator may initiate degrading of the inner wall by scraping it. In some embodiments, the inner wall may be scraped when the handle assembly 310 is drawn proximally, resulting in the distal disruptor tip 317 longitudinally scraping the inner wall of the targeted vessel. In other embodiments, the inner wall of the targeted vessel can be scraped when the handle assembly 310 is displaced proximally and manually rotated. In still other embodiments, the operator may push the control button 318a to activate rotation of the motor 343 and the distal disruptor tip 317. The operator can also simultaneously proximally displace the handle assembly 310 manually to longitudinally scrape the inner wall. Prior to, simultaneously with, or subsequent to scraping of the inner wall of the targeted vessel, the operator may press the control button 318b to activate the automatic injector 380 and start the flow of a sclerosant through the fluid transfer tubing 371 and out of the distal end of the intraluminal member 325.

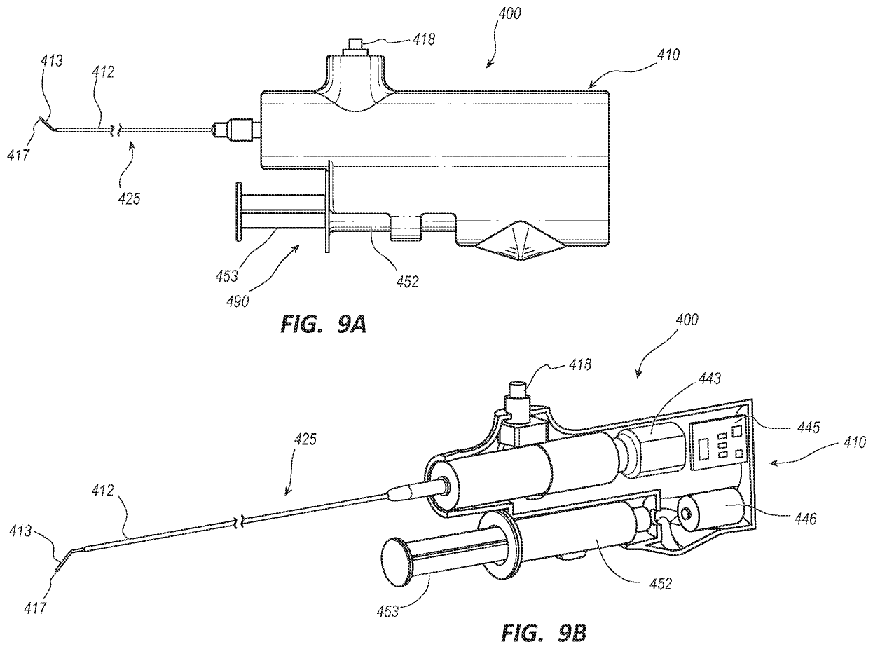

FIGS. 9A and 9B illustrate another embodiment of a vascular treatment device 400. The vascular treatment device 400 may include a handle assembly 410. The handle assembly 410 may include an intraluminal member 425 extending from a distal end of the handle assembly 410. The intraluminal member 425 may include a disruptor 413 disposed within a sheath 412. The handle assembly 410 may further include a motor 443, a control circuit 445, a power source 446, a control button 418, and a manual injector 490. The power source 446 may include one or more rechargeable or non-rechargeable batteries. The power source 446 may be electrically connected to the control circuit 445. The control circuit 445 may be in the form of a printed circuit board. The control circuit 445 may be electrically connected to the motor 443 and the control button 418.

The motor 443 may be operably coupled to a proximal end of the disruptor 413 and configured to rotate the disruptor 413 in both directions. In certain embodiments, the motor 443 may linearly translate the disruptor 413 distally relative to the intraluminal member 412 to expose a distal disruptor tip 417 of the disruptor 413 beyond a distal end of the sheath 412. In some embodiments, the motor 443 may be coupled to a proximal end of the sheath 412 to linearly translate the sheath 412 proximally relative to the disruptor 413 such that the distal disruptor tip 417 of the disruptor 413 is exposed beyond the distal end of the sheath 412.

As depicted in the illustrated embodiment, the manual injector 490 may include a syringe body 452 and a plunger 453 slidingly disposed within the syringe body 452. The manual injector 490 may be coupled to the handle assembly 410 such that the syringe body 452 may be oriented parallel to and radially offset from a longitudinal axis extending proximally from the intraluminal member 412. The plunger 453 may be directed distally.

In operating the vascular treatment device 400, the operator may create an incision or use an access sheath and insert the elongate intraluminal member 425 into a vessel that is targeted for treatment. The operator may position a distal end of the intraluminal member 425 at the place where the treatment of the vessel will commence and deploy the disruptor 413. The operator can depress the control button 418 to activate the motor to linearly displace the sheath 412 proximally such that distal disruptor tip 417 of the disruptor 413 is exposed from the distal end of the sheath 412 and forced towards, against, or to communicate with an inner wall of the targeted vessel.

The operator may initiate degrading of the inner wall of the targeted vessel by scraping it. In some embodiments, the inner wall of the targeted vessel may be scraped when the handle assembly 410 is displaced proximally, resulting in the distal disruptor tip 417 longitudinally scraping the inner wall of the targeted vessel. In other embodiments, the inner wall of the targeted vessel can be scraped when the handle assembly 410 is displaced proximally and manually rotated. In still other embodiments, the operator may push the control button 418 to activate rotation of the motor 443 and the distal disruptor tip 417. The operator can simultaneously proximally displace the handle assembly 410 to longitudinally scrape the inner wall. Prior to, simultaneously with, or subsequent to scraping of the inner wall of the targeted vessel, the operator may apply a proximally directed force to the plunger 453 to cause the flow of a sclerosant fluid from the syringe body 452 and through the intraluminal member 425. In other words, the plunger 453 is displaced by the operator in the same direction that the handle assembly 410 is displaced to scrape the vessel. This configuration may provide a more intuitive use of the vascular treatment device 400.

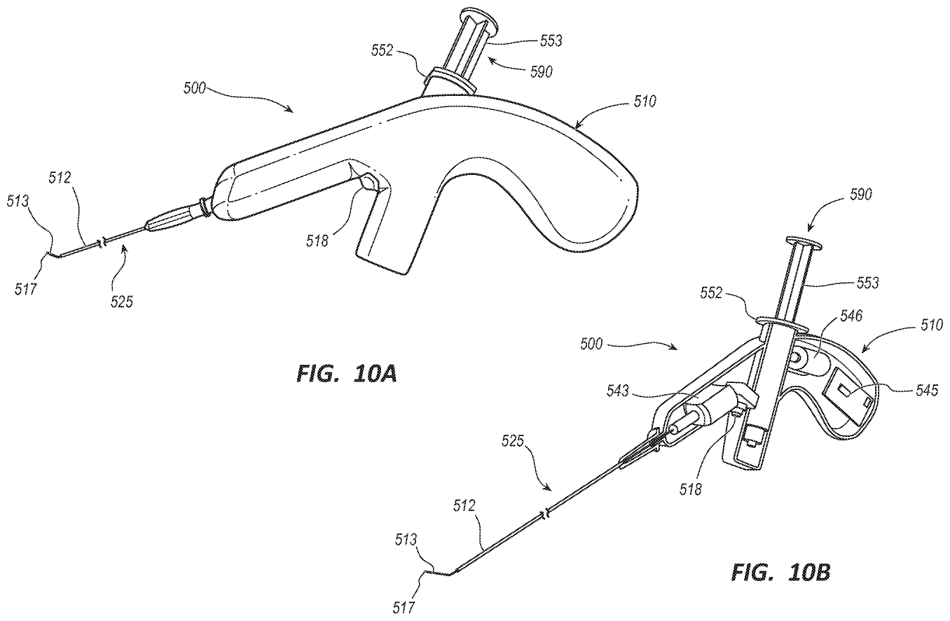

FIGS. 10A and 10B illustrate an embodiment of the vascular treatment device 500. The vascular treatment device 500 may include a handle assembly 510. The handle assembly 510 may include an intraluminal member 525 extending from a distal end of the handle assembly 510. The intraluminal member 525 may include a disruptor 513 disposed within a sheath 512. The handle assembly 510 may further include a motor 543, a control circuit 545, a power source 546, a control button 518, and a manual injector 590. The power source 546 may include one or more rechargeable or non-rechargeable batteries. The power source 546 may be electrically connected to the control circuit 545. The control circuit 545 may be in the form of a printed circuit board. The control circuit 545 may be electrically connected to the motor 543 and the control button 518.

The motor 543 may be operably coupled to a proximal end of the disruptor 513 and configured to rotate the disruptor 513 in both directions. In certain embodiments, the motor 543 may linearly translate the disruptor 513 distally relative to the sheath 512 to expose a distal disruptor tip 517 of the disruptor 513 beyond a distal end of the sheath 512. In some embodiments, the motor 543 may be coupled to a proximal end of the sheath 512 to linearly translate the sheath 512 proximally relative to the disruptor 513 such that the distal disruptor tip 517 of the disruptor 513 is exposed beyond the distal end of the sheath 512.

As depicted in the illustrated embodiment, the manual injector 590 may include a syringe body 552 and a plunger 553 slidingly disposed within the syringe body 552. The manual injector 590 may be coupled to the handle assembly 510 such that the syringe body 552 and plunger 553 may be oriented proximally at an acute angle relative to a longitudinal axis of the handle assembly 510.

In operating the vascular treatment device 500, the operator may create an incision or use an access sheath and insert the elongate intraluminal member 512 and the disruptor 513 into a vessel that is targeted for treatment. The operator may position a distal end of the intraluminal member 512 at a place where the treatment of the vessel will commence and then deploy the disruptor 513. For example, the operator can depress the control button 518 to activate the motor 543 to linearly displace the intraluminal member 512 proximally such that the distal disruptor tip 517 of the disruptor 513 is exposed from the distal end of the intraluminal member 512 and forced towards, against, or to communicate with an inner wall of the targeted vessel. In some embodiments, the operator can depress the control button 518 to activate the motor 543 to linearly displace the disruptor 513 distally such that the distal disruptor tip 517 of the disruptor 513 is exposed from the distal end of the intraluminal member 512 and forced towards, against, or to communicate with an inner wall of the vessel.

The operator may initiate degrading of the inner wall of the targeted vessel by scraping it. In some embodiments, the inner wall of the targeted vessel may be scraped when the handle assembly 510 is displaced proximally resulting in the distal disruptor tip 517 longitudinally scraping the inner wall of the targeted vessel. In other embodiments, the inner wall of the targeted vessel can be scraped when the handle assembly 510 is displaced proximally and manually rotated. In still other embodiments, the operator may push the control button 518 to activate rotation of the motor 543 and the distal disruptor tip 517. The operator can simultaneously proximally displace the handle assembly 510 to longitudinally scrape the inner wall. Prior to, simultaneously with, or subsequent to scraping of the inner wall of the targeted vessel, the operator may apply a distally directed force to the plunger 553 to cause the flow of a sclerosant fluid from the syringe body 552 and through the intraluminal member 512. In other words, the plunger 553 may be displaced by the operator in the opposite direction that the handle assembly 510 is displaced to scrape the vessel.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. An apparatus for occluding a vein, comprising:
a handle assembly comprising an intraluminal member extending distally from a distal end of the handle assembly, wherein the intraluminal member comprises:
a sheath; and
a disruptor disposed within the sheath;
a control module; and
a conduit member disposed between the handle assembly and the control module.

2. The apparatus of claim 1, wherein the handle assembly further comprises any one of a motor, an automatic injector, a reservoir, a control button, a control circuit, a status indicator, a power source, or any combination thereof.

3. The apparatus of claim 1, wherein the control module comprises any one of a motor, an automatic injector, a reservoir, a control button, a control circuit, a status indicator, a power source, or any combination thereof.

4. The apparatus of claim 1, wherein the control module is operably coupled to the handle by the conduit member.

5. The apparatus of claim 3, wherein the conduit member comprises a drive shaft, wherein a first end of the drive shaft is coupled to a proximal end of the disruptor and a second end of the drive shaft is coupled to the motor.

6. The apparatus of claim 5, wherein the drive shaft is flexible.

7. The apparatus of claim 3, wherein the conduit member comprises a fluid transfer tubing, wherein a first end of the fluid transfer tubing is in fluid communication with the reservoir and a second end of the fluid transfer tubing is in fluid communication with the intraluminal member.

8. The apparatus of claim 2, wherein the control button is configured to activate any one of the motor, the automatic injector, or any combination thereof.

9. The apparatus of claim 3, wherein the control button is configured to activate any one of the motor, the automatic injector, or any combination thereof.

10. The apparatus of claim 1, wherein the control module is configured to be disposed remotely from the handle assembly during a vein occlusion procedure.

11. The apparatus of claim 1, wherein the handle assembly further comprises a slot having a slide mounted therein.

12. The apparatus of claim 11, wherein the slide is coupled to the sheath, wherein the slide is moved in a proximal direction to pull back the sheath and expose a distal tip of the disruptor.

13. The apparatus of claim 11, wherein the slide is coupled to the disruptor, wherein when said slide is moved in a distal direction the disruptor is moved from within the sheath to expose a distal tip of the disruptor.

14. The apparatus of claim 1, wherein the disruptor, comprises:
an elongate wire having a distal end; and
a disruptor tip coupled to the distal end of the elongate wire, wherein the disruptor tip comprises:
at least two flexible arms comprising a proximal end and a distal end, first, second, and third straight segments disposed between the proximal end and the distal end, and a first bend disposed between the first and second straight segments, and a second bend disposed between the second and third straight segments, and
a disruptor head coupled to each one of the distal ends of the flexible arms.

15. The apparatus of claim 14, wherein the disruptor head comprises any one of an atraumatic configuration, a traumatic configuration, and a combination thereof.

16. A method of occluding a vessel, comprising:
obtaining a vessel occluding device comprising a handle assembly, a control module, and a conduit member, wherein the handle assembly comprises an intraluminal member comprising a sheath and a vessel disruptor;
inserting the intraluminal member into the vessel;
deploying the vessel disruptor;
disrupting a wall of the vessel with the vessel disruptor; and
injecting a sclerosant fluid into the vessel.

17. The method of claim 16, wherein the handle assembly further comprises any one of a motor, an automatic injector, a reservoir, a control button, a control circuit, a status indicator, a power source, or any combination thereof.

18. The method of claim 16, wherein the control module comprises any one of a motor, an automatic injector, a reservoir, a control button, a control circuit, a status indicator, a power source, or any combination thereof.

19. The method of claim 17, further comprising depressing the control button to activate any one of the motor, the automatic injector, or any combination thereof.

20. The method of claim 1, further comprising positioning the control module remotely from the handle assembly.
